(19) United States
(12) Patent Application Publication
(10) Pub. No.: US 2025/0059130 A1
(43) Pub. Date: Feb. 20, 2025

(54) PRODUCING PARACETAMOL FROM BIOMASS-DERIVED P-HYDROXYBENZOIC ACID AND P-HYDROXYBENZAMIDE

(71) Applicant: Wisconsin Alumni Research Foundation, Madison, WI (US)

(72) Inventors: John Ralph, Madison, WI (US); Steven Karlen, Madison, WI (US); Vitaliy Timokhin, Madison, WI (US); Canan Sener, Madison, WI (US)

(73) Assignee: Wisconsin Alumni Research Foundation, Madison, WI (US)

(21) Appl. No.: 18/802,453

(22) Filed: Aug. 13, 2024

Related U.S. Application Data
(60) Provisional application No. 63/519,392, filed on Aug. 14, 2023.

(51) Int. Cl. C07C 231/10; C07C 213/02; C07C 231/02; C07C 231/24

(52) U.S. Cl. CPC C07C 231/10; C07C 213/02; C07C 231/02; C07C 231/24

(57) ABSTRACT

A method for producing paracetamol from renewable biological feedstocks. The method comprises isolating p-hydroxybenzoic acid (pHBA) or a mixture of pHBA and p-hydroxybenzamide (pHBAm) from biomass. One version of the method comprises converting pHBA to pHBAm, subsequently to p-aminophenol (pAmP), and subsequently to paracetamol. The yield can be increased by recycling unreacted pHBAm to feed into the amide to amine conversion step. In a second version of the method, the phenolic hydroxyl group of pHBA or pHBAm is first protected. The subsequent reactions proceed as noted, with deprotection as the last step.

### CROSS-REFERENCE TO RELATED APPLICATIONS

Priority is hereby claimed to U.S. provisional application Ser. No. 63/519,392, filed Aug. 14, 2023, which is incorporated herein by reference.

### FEDERAL FUNDING STATEMENT

This invention was made with government support under DE-SC0018409 awarded by the US Department of Energy. The government has certain rights in the invention.

### BACKGROUND

Paracetamol (N-acetoxy-p-aminophenol; p-hydroxyacetanilide; N-(4-hydroxyphenyl)-acetamide) is one of the most heavily produced pharmaceuticals in the world. Annual global consumption (2022) is estimated to be roughly 16,250 metric tons valued at $130 million. FIG. 1 illustrates several prior art synthetic routes to paracetamol, including a route developed to utilize depolymerized lignin as the biomass source. The precursor to paracetamol in most synthetic routes is p-aminophenol, which itself has an estimated global market of ˜700 million tons valued at $1.4 billion (2022). In the scientific literature regarding methods to make paracetamol, see the following: E. Blondiaux et al., Bio-based aromatic amines from lignin-derived monomers. ACS Sustain. Chem. Eng. 7, 6906-6916 (2019); R. Joncour et al., Amidation of phenol derivatives: a direct synthesis of paracetamol (acetaminophen) from hydroquinone. Green Chem. 16, 2997-3002 (2014); J. Park et al., Production of active pharmaceutical ingredients (APIs) from lignin-derived phenol and catechol. Green Chem. 23, 7488-7498 (2021); H. Y. Chuang et al., Redox-Neutral Selenium-Catalysed Isomerisation of para-Hydroxamic Acids into para-Aminophenols. Angew. Chem., Int. Ed. 60, 13778-13782 (2021); M. Guidi et al., Combining radial and continuous flow synthesis to optimize and scale-up the production of medicines. React. Chem. Eng. 6, 220-224 (2021); J. D. Tibbetts et al., Sustainable syntheses of paracetamol and ibuprofen from biorenewable β-pinene. ChemSusChem, e202300670: 202300671-202300610 (2023).

The patent literature likewise describes several synthetic routes to make paracetamol. See, for example, U.S. Pat. No. 9,102,589, issued Aug. 11, 2015, to Krishna et al. This patent describes a continuous process for preparing primary and secondary N-acetylated aromatic amines via reactive distillation.

U.S. Pat. No. 5,856,575, issued Jan. 5, 1999, to Goinathan et al. This patent describes a route from hydroquinone to paracetamol. The fundamental steps include reacting a phenol with an amide in the presence of a solid catalyst composite material containing a heteropoly acid at a temperature in the range of 100 to 400° C. and separating the N-acetamino derivative from the products of the reaction.

See also U.S. Pat. No. 4,524,217, issued Jun. 18, 1985, to Davenport et al. In this approach, N-acyl-hydroxy aromatic amines (e.g., N-acetyl-p-aminophenol) are prepared by reacting a hydroxy aromatic ketone (e.g., 4-hydroxyacetophenone) with a hydroxylamine salt and a base to obtain the ketoxime of the ketone (e.g., 4-hydroxyacetophenone oxime), and then subjecting the ketoxime to a Beckmann rearrangement in the presence of a catalyst to form the corresponding N-acyl-hydroxy aromatic amine.

U.S. Pat. No. 2,998,450 issued Aug. 29, 1961, to Wilbert and De Angelis describe a route from nitrobenzene to paracetamol. In this route, nitrobenzene is electrolytically reduced to yield p-aminophenol. The p-aminophenol is acetylated to yield N-acetyl-p-aminophenol.

### SUMMARY

Producing commodity chemicals from renewable biological feedstocks is a key aspect of the movement to wean manufacturing from fossil fuel dependence. As society shifts to alternative sources of energy and fuels, there is a concomitant effort to identify and optimize alternative sources of entry-level platform chemicals. Disclosed herein is a method for converting the p-hydroxybenzoates found in some biomass feedstocks into valuable commodity chemicals. The primary biomass-derived compounds are p-hydroxybenzoic acid (pHBA), p-hydroxybenzamide (pHBAm), and p-aminophenol (pAmP), the latter of which efficiently converts to paracetamol. An alternative route produces p-(methoxymethoxy) aniline, N-acetyl-p-(methoxymethoxy) aniline, and paracetamol. These compounds are high-value, drop-in, renewable platform chemicals that can be used to make biodegradable plastics, pigments, and pharmaceuticals.

Thus, disclosed herein is a method to produce paracetamol from biomass-derived p-hydroxybenzoic acid (pHBA). The method comprises:
(a) providing pHBA sourced from biomass;
(b) converting at least a portion of the pHBA into p-hydroxybenzamide (pHBAm);
(c) converting at least a portion of the pHBAm into p-aminophenol (pAmP); and
(d) converting at least a portion of the pAmP into paracetamol.

In step (b), at least a portion of the pHBA is first esterified and then contacted with ammonium hydroxide to yield pHBAm. In certain embodiments, at least a portion of the pHBA is esterified by contacting it with an alcohol.

In step (c), at least a portion of the pHBAm is converted into pAmP using a Hofmann rearrangement reaction. The Hofmann rearrangement reaction yields pAmP and unreacted pHBAm. The method further comprises separating at least a portion of the unreacted pHBAm from the pAmP, and recycling the unreacted pHBAm into step (c). In certain embodiments, at least a portion of the unreacted pHBAm is separated from the pAmP via liquid-liquid extraction. The liquid-liquid extraction may be conducted using water and ethyl acetate.

In step (d), at least a portion of the pAmP is converted into paracetamol by contacting the pAmP with acetic anhydride.

Steps (b), (c) and (d) may be conducted in a solvent comprising water or ethyl acetate (EtOAc).

In a second version of the method, the phenolic hydroxyl group is first protected. The reactions then proceed as noted, with deprotection as the last step. Thus, with protection of the phenolic hydroxyl group, the method includes the following steps:
(a) providing pHBA sourced from biomass;
(b) appending a protecting group to at least a portion of phenolic hydroxyl groups in pHBA to yield phenolic-protected pHBA;
(c) converting at least a portion of the phenolic-protected pHBA into phenolic-protected p-hydroxybenzamide (pHBAm);
(d) converting at least a portion of the phenolic-protected pHBAm into phenolic-protected p-aminophenol (pAmP);
(e) converting at least a portion of the phenolic-protected pAmP into phenolic-protected paracetamol; and
(f) deprotecting the phenolic-protected paracetamol to yield paracetamol.

In step (b), a methoxymethyl ether (MOM) protecting group may be appended to at least a portion of phenolic hydroxyl groups in pHBA.

In step (d), at least a portion the phenolic-protected pHBAm is converted into phenolic-protected pAmP using a Hofmann rearrangement reaction. The Hofmann rearrangement reaction yields phenolic-protected pAmP and unreacted phenolic-protected pHBAm. The method further comprises separating at least a portion of the unreacted phenolic-protected pHBAm from the phenolic-protected pAmP, and recycling the unreacted phenolic-protected pHBAm into step (d). In certain embodiments, at least a portion of the unreacted phenolic-protected pHBAm is separated from the pAmP via liquid-liquid extraction. The liquid-liquid extraction may be conducted using water and ethyl acetate.

In step (e), at least a portion of the phenolic-protected pAmP is converted into phenolic-protected paracetamol by contacting the pAmP with acetic anhydride.

Steps (b), (c), (d), (e), and (f) may be conducted in a solvent comprising water or ethyl acetate (EtOAc).

The pHBA can be isolated from biomass using any methods known in the art, including, but not limited to, mild alkaline treatment. Alternatively, treating the biomass with aqueous ammonium hydroxide produces a mixture of pHBA and pHBAm.

Thus, also disclosed herein is a method to produce paracetamol from biomass-derived p-hydroxybenzamide (pHBAm). The method comprises:
(a) providing pHBAm sourced from biomass;
(b) converting at least a portion of the pHBAm into p-aminophenol (pAmP); and
(c) converting at least a portion of the pAmP into paracetamol.

An alternative version of the method comprises:
(a) providing pHBAm sourced from biomass;
(b) appending a protecting group to at least a portion of phenolic hydroxyl groups in pHBAm to yield phenolic-protected pHBAm;
(c) converting at least a portion of the phenolic-protected pHBAm into phenolic-protected p-aminophenol (pAmP);
(d) converting at least a portion of the phenolic-protected pAmP into phenolic-protected paracetamol; and
(e) deprotecting the phenolic-protected paracetamol to yield paracetamol.

The objects and advantages of the disclosure will appear more fully from the following detailed description of the preferred embodiment of the disclosure made in conjunction with the accompanying drawings.

### BRIEF DESCRIPTION OF THE DRAWINGS

FIG. 1 depicts various synthetic routes to produce paracetamol.

FIG. 2 depicts synthetic routes to produce paracetamol from pHBA. Panel A: A four-step process to convert pHBA to paracetamol via the Hofmann rearrangement. Panel B: A five-step process uses a phenol protecting group (methoxymethyl ether, MOM) to stabilize the p-aminophenol intermediate. The process can also be initiated from pHBAm, removing one of the steps in the pathway.

FIG. 3A depicts a process scheme to produce pHBAm directly from biomass and convert the pHBAm to paracetamol in a continuous process using the Hofmann rearrangement to make p-aminophenol, liquid/liquid recovery, and recycling the unreacted pHBAm. Acetic anhydride is used to acetylate the amine to form paracetamol and neutralize the solution. Liquid/liquid extraction is used to isolate paracetamol in 41-57 wt % yield (Table 2).

FIG. 3B is a schematic diagram of a preferred version of the method disclosed herein. The diagram depicts the operational steps and chemical flow scheme used to convert biomass-derived pHBA to paracetamol.

FIG. 4 is a schematic illustrating the semi-batch production of pAmP and paracetamol from pHBAm. Solutions of pHBAm in 2 M NaOH and sodium hypochlorite (NaOCl) in 2 M NaOH were pumped independently into a T-union and then through a static mixer. The reaction mixture was then passed through a 2.0 mL reaction loop and dropped into a 2 M sodium bisulfite (NaHSO₃) quenching solution in an ice bath. Once the target reaction volume was dispensed, the collection flask was removed and the intermediate solution treated with acetic anhydride to form the target product (paracetamol).

FIG. 5. Plot showing how the [NaOCl]:[pHBAm] ratio impacts the conversion of pHBAm to p-aminophenol. The y-values are the relative percentage of three primary products: acetylated pHBAm, (di) acetylated aminophenol (paracetamol and diacetylated 4-aminophenol). The y-error bars represent the average of n=3 technical replicates the x-error bars represent the 3-5 wt % range of reagent grade NaOCl.

### DETAILED DESCRIPTION

### Abbreviations and Definitions

All references to singular characteristics or limitations of the disclosed method shall include the corresponding plural characteristic or limitation, and vice-versa, unless otherwise specified or clearly implied to the contrary by the context in which the reference is made. The indefinite articles “a” and “an” mean “one or more.”

All combinations of method steps disclosed herein can be performed in any order, unless otherwise specified or clearly implied to the contrary by the context in which the referenced combination is made. The disclosure provided herein suitably may be practiced in the absence of any element which is not specifically disclosed herein.

The method disclosed herein can comprise, consist of, or consist essentially of the essential elements and steps described herein, as well as any additional or optional ingredients, components, or limitations described herein or otherwise useful in organic chemistry. The disclosure provided herein suitably may be practiced in the absence of any element which is not specifically disclosed herein.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

The word “or” is defined inclusively and should be read as “and/or.”

Generally, reference to a certain element such as hydrogen or H is meant to include all isotopes of that element. For example, if an R group is defined to include hydrogen or H, it also includes deuterium and tritium. Compounds comprising radioisotopes such as tritium, C¹⁴, P³² and S³⁵ are thus within the scope of the present technology. Procedures for inserting such labels into the compounds of the present technology are readily apparent to those skilled in the art based on the disclosure herein.

The term “carboxylate” as used herein refers to a —COOH group.

The term “ester” as used herein refers to —COOR groups, in which R is a substituted or unsubstituted alkyl, cycloalkyl, alkenyl, alkynyl, aryl, aralkyl, heterocyclylalkyl, heterocyclyl, etc.

The term “amide” (or “amido”) includes C- and N-amide groups, i.e., —C(═O)NRR′, and —NRC(═O) R′ groups, respectively. R and R′ are independently hydrogen, or a substituted or unsubstituted alkyl, alkenyl, alkynyl, cycloalkyl, aryl, aralkyl, heterocyclylalkyl or heterocyclyl, etc.

The term “amine” (or “amino”) as used herein refers to —NRR′ groups, wherein R and R′ are independently hydrogen, or a substituted or unsubstituted alkyl, alkenyl, alkynyl, cycloalkyl, aryl, aralkyl, heterocyclylalkyl, heterocyclyl, etc.

The term “hydroxy” as used herein can refer to —OH or its ionized form, —O⁻. An “alcohol” contains at least one hydroxy group. Common primary alcohols include methanol, ethanol, 1-propanol, 1-butanol, isobutyl alcohol, etc. Common secondary alcohols include isopropyl alcohol, cyclohexanol, etc. Common tertiary alcohols include tert-butyl alcohol, tert-amyl alcohol, and the like.

A protecting group is any chemical moiety capable of selective addition to and removal from a reactive site to allow manipulation of a chemical entity at sites other than the reactive site. A host of protecting groups, and how to attach and remove them selectively, are known in the art and will not be described herein in any detail. See, for example, “Greene's Protective Groups in Organic Synthesis,” 5^{th} Edition; ISBN-13:978-1118057483, ©2014, John Wiley & Sons, Inc. Greene describes many nitrogen and oxygen protecting groups. Methoxymethyl ether (MOM) and methoxyethoxy ether (MEM), for example, are widely used as alcohol protecting groups. So too are silyl ethers, such as trimethylsilyl (TMS).

The term “contacting” refers to the act of touching, making contact, or of bringing to immediate or close proximity, including at the molecular level, for example, to bring about a chemical reaction, or a physical change, e.g., in a solution or in a reaction mixture.

An “effective amount” refers to an amount of a chemical or reagent effective to facilitate a chemical reaction between two or more reaction components, and/or to bring about a recited effect. Thus, an “effective amount” generally means an amount that provides the desired effect.

The term “solvent” refers to any liquid that can dissolve a compound to form a solution. Solvents include water and various organic solvents, such as hydrocarbon solvents, for example, alkanes and aryl solvents, as well as halo-alkane solvents. Examples include hexanes, benzene, toluene, xylenes, chloroform, methylene chloride, dichloroethane, and alcoholic solvents such as methanol, ethanol, propanol, isopropanol, and linear or branched (sec or tert) butanol, and the like. Aprotic solvents that can be used in the method include, but are not limited to perfluorohexane, α,α,α-trifluorotoluene, pentane, hexane, cyclohexane, methylcyclohexane, decalin, dioxane, carbon tetrachloride, freon-11, benzene, toluene, triethyl amine, carbon disulfide, diisopropyl ether, diethyl ether, t-butyl methyl ether (MTBE), chloroform, ethyl acetate, 1,2-dimethoxyethane (glyme), 2-methoxyethyl ether (diglyme), tetrahydrofuran (THF), methylene chloride, pyridine, 2-butanone (MEK), acetone, hexamethylphosphoramide, N-methylpyrrolidinone (NMP), nitromethane, dimethylformamide (DMF), acetonitrile, sulfolane, dimethyl sulfoxide (DMSO), propylene carbonate, and the like.

Ac₂O=acetic anhydride. EFB=empty fruit bunch (fibers, from African oil palm, Elaeis guineensis). EtOAc=ethyl acetate. GVL=gamma-valerolactone. MOM=methoxymethyl ether. NM6=NM6 hybrid poplar (Populus maximowiczii x nigra). pAmP=p-aminophenol. pHBA=p-hydroxybenzoic acid. Et-pHBA=ethyl p-hydroxybenzoate. pHABm=p-hydroxybenzamide. WCW=whole cell wall.

Producing Paracetamol from Renewable Biological Feedstocks

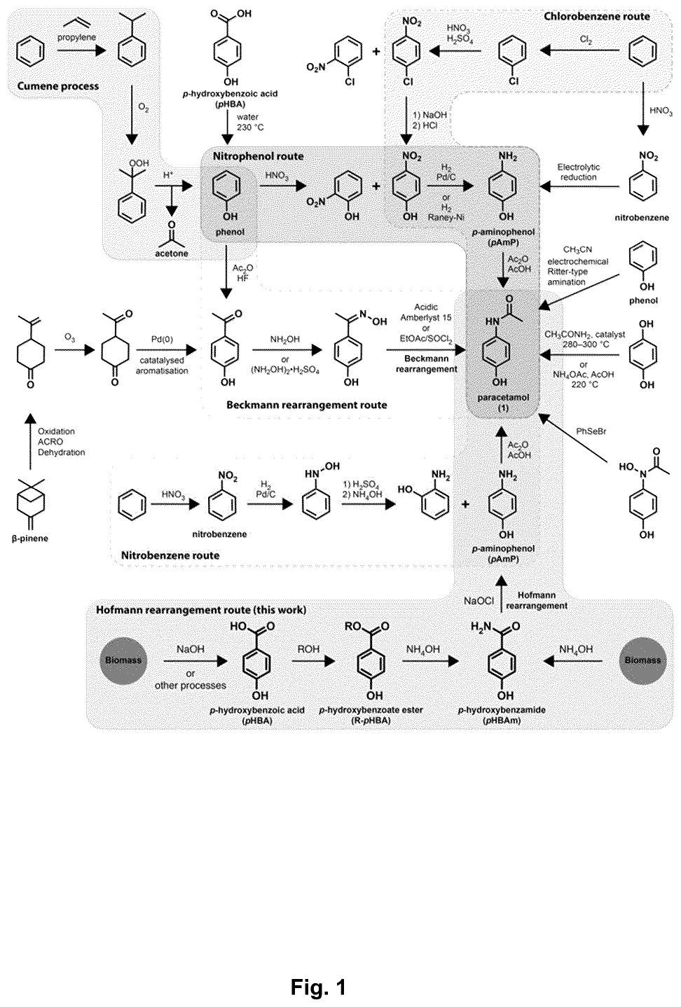

Utilizing biomass-derived chemicals to synthesize paracetamol or p-aminophenol requires at some point separating the target compounds from a complex mixture of co-products. At least conceptually, several of the known synthetic routes to paracetamol are adaptable to produce paracetamol from biomass-derived chemical feedstocks (see FIG. 1). But this would be possible only after one or more high-energy processes and purification operations to yield a suitably pure product. An economically feasible and environmentally sustainable route to paracetamol must also meet certain criteria: The process should use water as the primary solvent and green organic solvents when required (e.g., in liquid/liquid purification steps). The chemical precursor to paracetamol should be produced and isolated from lignocellulosic biomass using technologies proven to be scalable. The method should use low-cost, low-energy processes (e.g., counter-current extraction, crystallization, and distillation for low-boiling-point products).

A precursor chemical that meets these criteria is p-hydroxybenzoic acid (pHBA):

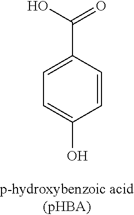

In the present method, the pHBA is esterified, converted to p-hydroxybenzamide (pHBAm) and, using the Hofmann rearrangement, transformed into p-aminophenol. pHBA is found in nature as a substructure of plant metabolites, including clades of angiosperms (flowering plants) from the Family Salicaceae (which includes Salix and Populus, e.g., willow, poplar, aspen, and cottonwood) and the Family Arecaceae (palms; e.g., coconut, carnauba, date, and oil palms). pHBA is produced by alkaline or acidic hydrolysis of the benzoate esters from these common plant species along with Aralia cordata in the Asterid clade. Depending on the species and tissue type, pHBA can account for up to 1.5 wt % of extracted woody biomass, e.g., 0.2-1.0 wt % of dried poplar wood and 1.0-1.5 wt % of the oil palm tree's empty fruit bunches (EFB). (L. de Vries et al., pHBMT1, a BAHD-family monolignol acyltransferase, mediates lignin acylation in poplar. Plant Physiol. 188, 1014-1027 (2022); F. Lu et al., Naturally p-hydroxybenzoylated lignins in palms. Bioenerg Res. 8, 934-952 (2015)).

Many lignocellulose deconstruction processes cleave the ester bond linking pHBA to the cell wall. In some cases, the process will convert the lignin and some of the metabolites to pHBA (See Zakzeski, et al., The catalytic valorization of lignin for the production of renewable chemicals. Chem. Rev. 110, 3552-3599 (2010); Sun, et al., Bright side of lignin depolymerization: Toward new platform chemicals. Chem. Rev. 118, 614-678 (2018); and Schutyser et al., Chemicals from lignin: an interplay of lignocellulose fractionation, depolymerisation, and upgrading. Chem. Soc. Rev. 47, 852-908 (2018)). In 2022, the global market for pHBA was ˜42,500 tons, and valued at ˜$83 million USD. (MarketWatch.Com, Jun. 15, 2023; marketwatch.com/press-release/4-hydroxybenzoic-acid-market-size-regional-status-and-outlook-2023-2030-2023-06-15). A single biorefinery processing 10,000 tons/day of poplar wood with a pHBA level of 1.2 wt % could produce 120 tons/day pHBA as a co-product and meet the 2022 global demand. Sustainable biorefinery design models indicate that, for viable replacement of petroleum-derived liquid fuels, there would need to be a network of smaller biorefineries feeding larger hub refineries to upconvert the products. Ideally the smaller refineries would be able to utilize similar processes to reduce the cost of design and construction. Identifying higher-value products with larger global markets (e.g., p-aminophenol and paracetamol) that can be produced from pHBA would enable more biorefinery clones to feed a larger hub and not saturate the global pHBA market. If the biorefineries were designed to utilize more than the cell-wall-bound pHBA and could instead (or additionally) use catalytic lignin depolymerization to convert a portion of the lignin to pHBA (e.g., converting half of the lignin, or about 10 wt % of the biomass, at a level of 1,200 tons/day), then it would be even more important to develop alternative product streams for pHBA.

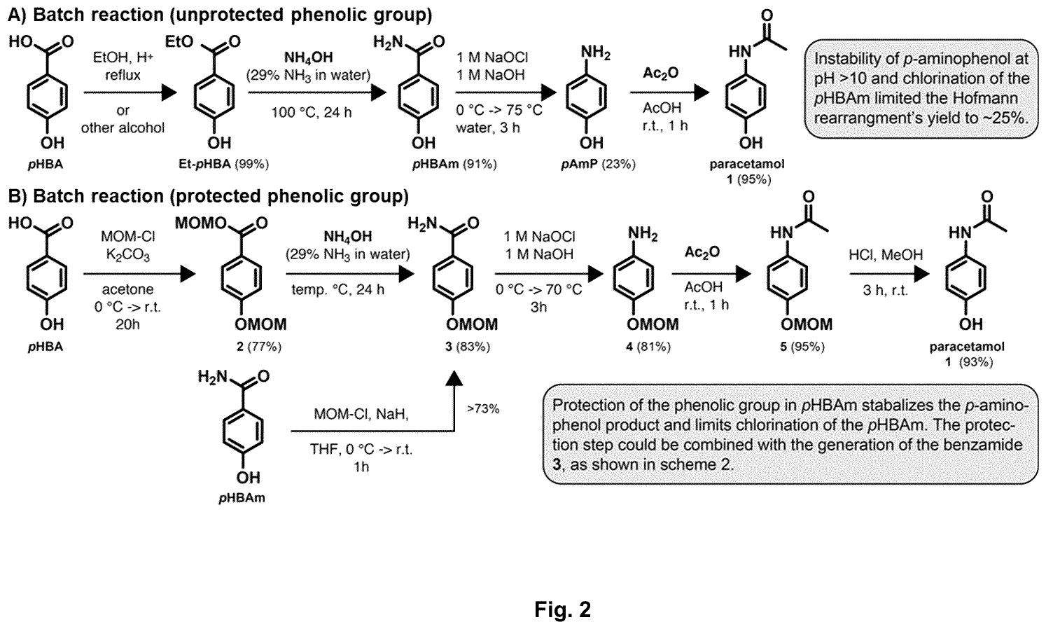

The pHBA can be isolated by cleaving it from lignin or whole-cell-wall (WCW) biomass using mild alkaline treatment, e.g., 0.2 M NaOH at 25° C. See Table 1. To isolate the liberated pHBA, the treatment liquor is separated from the solids, either before or after acidification to PH ˜1. Acidification of the aqueous solution causes some solubilized sugars and phenolics to precipitate, possibly requiring a second separation step. Once the supernatant is acidified the pHBA can be isolated either by concentration of the aqueous solution and crystallization or through liquid/liquid extraction using an organic solvent such as ethyl acetate (EtOAc). Removal of the organic solvent and recrystallization from hot water results in a pure (>95% by NMR analysis) off-white pHBA powder. The pHBA can then be converted to an ester (e.g., methyl p-hydroxybenzoate) and to pHBAm using aqueous ammonium hydroxide (e.g., 29% NH₄OH) at 100-140° C. (FIG. 2, panel A); direct amidation of the p-hydroxybenzoic acid does not proceed under these conditions.

Alternatively, treating the biomass lignin or WCW biomass with aqueous ammonium hydroxide liberates a mixture of pHBAm and pHBA from the lignin-bound pHBA esters. At low temperatures, pHBA (the free acid) predominates. At higher reaction temperatures (e.g., ˜140° C.), the esters are converted to the amide with up to an impressive 99% selectivity. (See Table 1.) The yield of either the pHBA or pHBAm is dependent on the feedstock, with the yields from 0.2 M NaOH hydrolysis at 25° C. closely matching those obtained from 29% NH₄OH at 140° C. This is demonstrated here using NM6 poplar, a Populus maximowiczii x nigra hybrid, from which lignin was isolated via a gamma-valerolactone (GVL) process producing pHBAm in 3.1 wt % yield (or from WCW biomass in 0.5 wt % yield). Oil palm empty fruit bunches (EFB) were also used as a biomass feedstock, which yielded 1.4 wt % pHBA (free acid) or 1.2 wt % pHBAm (amide).

TABLE 1
Production of pHBA and pHBAm from debarked poplar wood and oil palm EFBs.
[pHBAm]:[pHBA] ratio determined from ¹H NMR of product mixture.
pHBA was isolated using 0.2M sodium hydroxide to cleave pHBA esters, followed
by an acidic digestion to cleave phenolic glucosides. The pHBAm was isolated
using 29% ammonium hydroxide to generate the pHBAm from the pHBA esters.

| | Biomass | T | Reaction | 0.2M NaOH | Mass | pHBA | |
|---|---|---|---|---|---|---|---|
| Run | type | (° C.) | Time (h) | (mL) | (g) | (wt %) | [pHBAm]:[pHBA] |
| Oil Palm EFB | WCW | 25² | 24 | 10.0 | 1.75 | 1.4 | 0:100 |
| NM6 poplar | WCW | 25² | 24 | 10.0 | 5.0 | 0.5 | 0:100 |
| NM6 poplar | lignin | 25² | 24 | 10.0 | 5.0 | 4.5 | 0:100 |
| | Biomass | T | Reaction | 15.35M (29%) | Mass | pHBAm | |
| Run | type | (° C.) | Time (h) | NH₄OH (mL) | (g) | (wt %) | [pHBAm]:[pHBA] |
| Oil Palm EFB | WCW | 140 | 24 | 10.0 | 0.50 | 1.2 | 99:1 |
| NM6 poplar | WCW | 140² | 24 | 10.0 | 0.50 | 0.5 | 99:1 |
| NM6 poplar | lignin | 140² | 24 | 10.0 | 0.20 | 3.1 | 99:1 |
| NM6 poplar | lignin | 140² | 24 | 10.0 | 0.10 | | 98:2 |
| NM6 poplar | lignin | 130² | 24 | 10.0 | 0.10 | | 94:6 |
| NM6 poplar | lignin | 120² | 24 | 10.0 | 0.10 | | 67:33 |
| NM6 poplar | lignin | 100¹ | 24 | 10.0 | 0.10 | | 50:50 |
| NM6 poplar | lignin | 80¹ | 24 | 10.0 | 0.10 | | 43:57 |
| NM6 poplar | lignin | 60² | 24 | 10.0 | 0.10 | | 23:77 |
| NM6 poplar | lignin | 60¹ | 24 | 10.0 | 0.10 | | 30:70 |
| NM6 poplar | lignin | 40¹ | 24 | 10.0 | 0.10 | | 13:87 |
| NM6 poplar | lignin | 25² | 24 | 10.0 | 0.10 | | 11:89 |
| NM6 poplar | lignin | 25¹ | 24 | 10.0 | 0.10 | | 15:85 |

¹Reactions were performed in glass pressure vessels.
²Reactions were performed in stainless steel pressure vessels.

The yield of paracetamol in the Hofmann rearrangement of pHBAm to paracetamol under batch reaction conditions was limited to ˜25%. See FIG. 2, panel A. This was due to several factors: the reactivity of p-aminophenol under the reaction conditions (2 M NaOH, pH >10); from side-reactions (chlorination of the electron-rich aromatic ring by the sodium hypochlorite); and from competing reactions with the unreacted pHBAm. Varying the concentration of NaOCl and NaOH, the reaction temperature, and the reaction time altered the percent conversion, but did not increase the yield of product above ˜25%. When the hydroxy group of pHBAm was protected, however (e.g., as the MOM ether, producing p-(methoxymethoxy)benzamide, pMOMBAm, 3) the Hofmann rearrangement quantitatively formed the MOM-protected p-aminophenol (4) and no chlorination or polymerization products were observed. Acetylation of amine 4 followed by acid-catalyzed deprotection of the MOM group on compound 5 generated paracetamol with high overall yields. See FIG. 2, panel B.

Depending on the importance placed on atom economy, the MOM protection could be performed directly on the pHBAm (e.g., using methoxymethyl chloride and sodium hydride) or the pHBA could be reacted with two equivalents of methoxymethyl chloride to both protect the phenolic group and form the ester required for the amidation reaction (FIG. 2, panel B). This route produces a secondary target product p-(methoxy)benzylamine, which is itself a commodity chemical used in polymers, dyes, and resins.

TABLE 2
Continuous-flow synthesis of paracetamol¹.

| | | | Yield | Product balance |
|---|---|---|---|---|
| | | Recovered | paracetamol, | pHBAm + paracetamol |
| Entry | [NaClO]:[pHBAm] | pHBAm (mol %) | (mol %) | (mol %) |
| 1 | 5.5 | 12 | 47 | 59 |
| 2 | 3.8 | 28 | 53 | 81 |
| 3 | 3.3 | 31 | 50 | 81 |
| 4 | 2.8 | 30 | 57 | 87 |
| 5 | 1.1 | 50 | 41 | 91 |

¹Reaction conditions: Flow rate: 1.5 mL/min of A: 0.182M pHBAm in 2M NaOH; 1.5 mL/min of B: 1.00M NaOCl in 2M NaOH; total flow rate 3 mL/min; sample loop 2 mL; residence time 40 s; collection time 20 min; reaction temperature 25° C.; quench at 0° C. with 2M NaHSO₃.

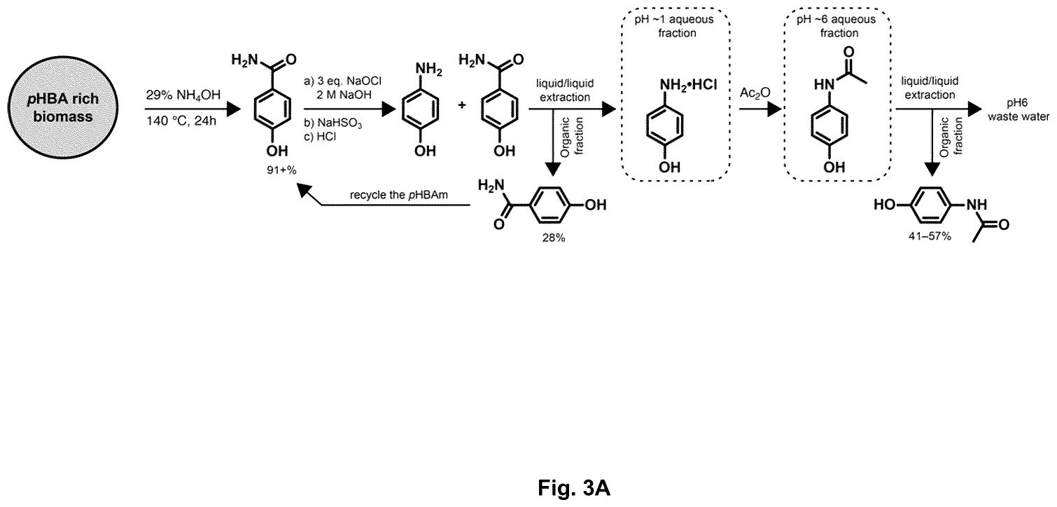

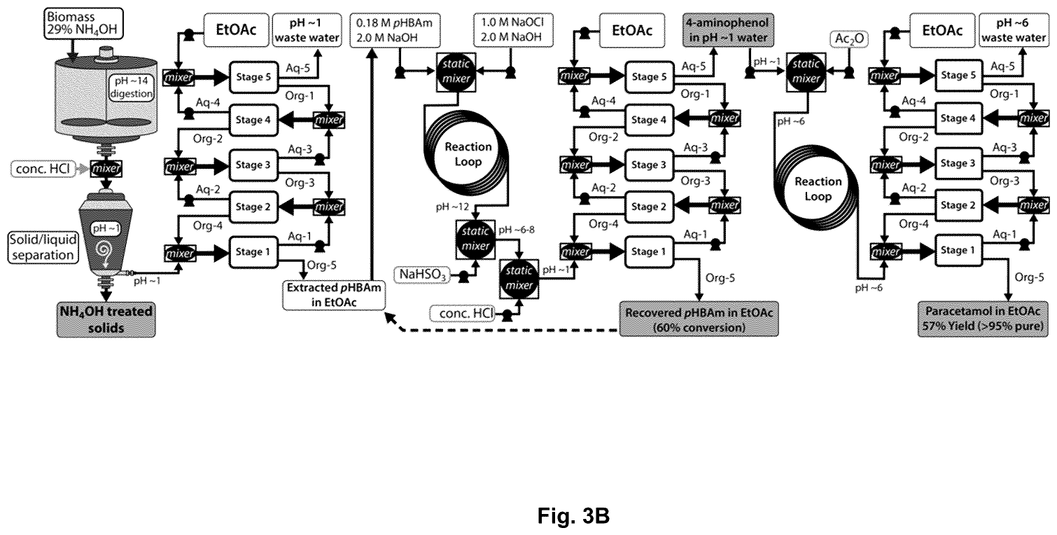

By transitioning to a continuous-flow-chemistry technology (FIGS. 3A and 3B), product stability was improved, and side reactions were suppressed. Tuning the residence time of the reaction and the ratio of [NaOCl]:[pHBAm] allowed for optimization of the conversion of pHBAm by the Hofmann rearrangement and the yield of paracetamol (following acetylation), and minimizing the loss of p-aminophenol to degradation. See Table 2. To stabilize the product mixture, excess NaOCl was quenched with sodium bisulfite (NaHSO₃); similar reagents (sodium dithionite) could also be used. The resulting aqueous reaction mixture (pH ˜6) containing p-aminophenol, pHBAm, sodium chloride, sodium sulfate, and sodium bisulfite was acidified to pH ˜1 with, hydrochloric or sulfuric acid. The unconverted pHBAm was recovered by liquid-liquid extraction with ethyl acetate (with or without membrane assistance). The pHBAm was extracted into the organic phase, whereas the protonated p-aminophenol remained in the aqueous phase. The p-aminophenol was then acetylated using acetic anhydride (Ac₂O), which also adjusted the solution to PH ˜6. The paracetamol was then isolated by liquid-liquid extraction using ethyl acetate (with or without membrane assistance). Removal (with recovery) of the solvent yielded the paracetamol product as a white solid. Paracetamol yields ranged from 41-57% yield depending on the [NaOCl]:[pHBAm] ratio. See Table 2. The crude paracetamol can be purified further by recrystallization from water or via more extensive purification strategies (e.g., silica gel chromatography) as required for downstream applications.

The continuous flow process shown in FIG. 3B uses EtOAc as the organic solvent and NaOCl as the oxidant. The conversion of pHBAm to p-aminophenol was adjusted to 57%, with recovery of pHBAm at 30% (Table 2, entry 4). One of the advantages of this stepwise purification scheme is the ability to recycle the recovered pHBAm back through the Hofmann rearrangement reaction. Doing so increases the overall conversion of pHBAm to p-aminophenol. Under these conditions, and recycling the unreacted pHBAm, the overall yield for pHBAm to paracetamol was ˜90%.

Utilizing small (˜10,000 metric ton/day) biorefineries operating with dilute ammonia or mild alkaline pretreatment to co-produce 120 metric ton/day pHBA or pHBAm during the detoxification of the aqueous pretreatment liquor adds value to the pretreatment process. The conversion of some of the phenolics in the pretreatment liquor to p-aminophenol and paracetamol expands the size and scale of the product market from ˜$85 million to over $1.5 billion. This greatly increases the viability of constructing a network of small biorefineries that can feed large biomass conversion hubs-thereby further enabling the global transition from petrochemical-sourced fuels and chemicals to processes using renewable, sustainable materials.

### Examples

The following Examples are included solely to provide a more complete description of the method disclosed and claimed herein.

### Materials and Methods:

All commercially available reagents and solvents were used without further purification unless otherwise stated. Flash-column chromatography of the products was carried out using silica gel 60A, particle size 230-400 micron (Merck/Millipore Sigma, Burlington, Massachusetts, USA). Analytical thin-layer chromatography (TLC) was performed on DC-Alufolien Kieselgel 60F₂₅₄ 0.2 mm plates (Merck), and compounds were visualized by UV fluorescence at 254 and 365 nm. ¹H, ¹³C, 2D HSQC, and 2D HMBC NMR spectra were recorded in deuterated solvents on a Bruker Biospin (Billerica, Massachusetts, USA) AVANCE 500 or NEO 700 spectrometers fitted with cryogenically-cooled gradient probes. Spectral processing used Bruker's Topspin 4 (Mac) software. The central residual solvent peaks were used as an internal references: acetone-d₆ δ_{H}=2.04 ppm, δ_{C}=29.80 ppm; DMSO-d₆ δ_{H}=2.49 ppm, δ_{C}=39.50 ppm; CDCl₃ δ_{H}=7.24 ppm, δ_{C}=77.23 ppm; methanol-d₄ δ_{H}=3.31 ppm, δ_{C}=49.15 ppm. Coupling constants (J-values) are given in hertz (Hz), and the data are reported in standard form as follows: chemical shift in ppm (number of protons, splitting pattern, coupling constant(s) where applicable, assignment). Spectra were assigned with the help of literature values for similar compounds. ¹H NMR spectra and peak integrals were used to determine ratios of the products in the reaction mixture. The reactions were performed in round-bottom flasks (25-500 mL), or 60 mL sealed glass pressure vessels (Ace Glass Inc., Vineland, New Jersey, USA, P/N: 8648-32) with a #15 polytetrafluoroethylene (PTFE) seal plug (Ace Glass Inc., P/N: 8648-20) at temperature below 100° C., or a 25 mL stainless steel pressure vessel (#1487, Parr Instrument Company, Moline, Illinois, USA) at temperatures above 100° C. Continuous-flow reactions were conducted using Shimadzu LC10 pumps, 25 μL binary tee static mixer (Analytical Scientific Instruments US, Inc., Richmond California, USA, P/N: 403-0025HP), using Chrom Tech (Apple Valley, Minnesota, USA) 10 mL (P/N: SL10KCW), 2 mL (P/N: SL2KCUW), or 1 mL (P/N: SL1KCUW) stainless steel sample loops, fitted with in-line check valves (P/N: CV-3340) and using 1/16×0.02 in. PFA tubing (P/N: 1512L).

### Plant Materials:

The NM6 hybrid poplar (Populus maximowiczii x nigra) was debarked, chipped, dried, and fractionated to pass through a 5 mm round hole on a shaker table. The African oil palm (Elaeis guineensis) empty fruit bunch (EFB) fibers were obtained from the Ladang Tai Tak Sdn. Bhd. Palm oil mill in Kota Tinggi, Johor, Malaysia. The EFB fibers were steam-treated (45 psi, 130° C., 70 min) to remove the fruitlets and then shredded into fibers (10-15 cm in length).

The dried biomass was milled to a fine powder using a Retsch MM-400 shaker-mill (Verder Scientific Inc., Newtown, Pennsylvania, USA). The biomass (1-2 g biomass per jar) was loaded into 50 mL stainless steel jars with one 25 mm stainless steel ball-bearing and milled at 30 Hz for 3 min. The resulting fine power, in 20 batches, was pooled to make large, homogenized pools of biomass for the whole-cell-wall studies.

### Production of NM6 Poplar Lignin Using the GVL-Extraction Method:

Lignin was extracted from NM6 poplar using the gamma-valerolactone (GVL) pretreatment process described previously. (D. M. Alonso et al., Increasing the revenue from lignocellulosic biomass: Maximizing feedstock utilization. Sci. Adv. 3, e1603301: 1603301-1603307 (2017); J. M. Perez et al., Integrating lignin depolymerization with microbial funneling processes using agronomically relevant feedstocks. Green Chem. 24, 2795-2811 (2022).)) Briefly, 185 g of biomass was mixed in 1665 g of a 100 mM sulfuric acid solution of GVL and water (90:10 wt/wt). The solution was incubated at room temperature (23° C.) overnight to allow the air trapped in the biomass to escape. The mixture was then loaded into a 2 L semi-batch Parr reactor (Parr Instrument Company, Moline, Illinois, USA) and heated to 90° C. for 90 min. Fresh GVL/water (2 L, 90:10, wt/wt, without acid) was then pumped into the reactor at 40 mL/min to elute the reaction solution through a glass fiber filter plug at the bottom of the reactor and a water bath that cooled to eluent to ˜50° C. The first 1 L of GVL/water eluent was further cooled to room temperature (˜23° C.) and then the lignin was precipitated by dilution with water (1:9 v/v, eluent-to-water). The resulting lignin suspension (˜10 L) was allowed to settle for two days prior to decanting off the top ˜9 L of the aqueous solution. The remaining ˜1 L of solution containing the extracted lignin was centrifuged to a lignin pellet and the supernatant was decanted off. The pellet was washed three times by resuspending the lignin in hot RO (reverse osmosis) water, reformation of the lignin pellet, and decanting the supernatant. After the third hot RO water washing step, the wet lignin pellet was freeze-dried to produce a light brown lignin powder that was used without further purification.

Production of p-Hydroxybenzoic Acid (pHBA) from Biomass and Lignin:

Production of pHBA from poplar NM6 GVL lignin. The NM6 poplar lignin (5.0 g) was added into a 500 mL round-bottom flask containing a magnetic stir bar and fitted with a rubber stopper. Aqueous 0.2 M NaOH (300 mL) was added and the reaction mixture was stirred at room temperature for 24 h. After the mixture was adjusted from pH ˜12 to pH ˜1 using concentrated HCl (35 wt %). The mixture was centrifuged (8,000 rpm) at room temperature for 10 min, and the supernatant was decanted off. The residual lignin was washed with water (2×30 mL), the mixture was centrifuged again, and the supernatants were combined. The crude pHBA was extracted using EtOAc (3×100 mL), and the organic layers were combined, washed with brine (1×30 mL), dried over anhydrous sodium sulfate (Na₂SO₄), and filtered. Upon evaporation of the solvent in vacuo, the obtained white solid was subjected to column chromatography on silica gel with eluent EtOAc to obtain purified pHBA (231 mg, 4.6 wt %). ¹H NMR (700 MHz, DMSO-d₆) δ 6.80 (d, 2H, J=8.7 Hz, H_{3/5}), 7.77 (d, 2H, J=8.7 Hz, H_{2/6}), 10.20 (s, 1H, Ar—OH). ¹³C NMR (125 MHZ, DMSO-d₆) δ 167.23 (C₇), 161.65 (C₄), 131.59 (C_{2/6}), 121.37 (C₁), 115.16 (C_{3/5}).

Production of pHBA from poplar NM6 lignin by water hydrolysis (without using NaOH): The NM6 poplar lignin (100 mg) and DI water (5 mL) were combined in a 25 mL stainless steel pressure vessel containing a magnetic stir bar. The reactor was purged with argon and sealed. The reaction mixture was heated in an oil bath at 160° C. for 1 h and then removed from the bath and cooled to 0° C. in an ice bath. The reactor was opened and the pH of the mixture was ˜5. The mixture was transferred to falcon tube and centrifuged (8,000 rpm) for 5 min at room temperature, and the supernatant was decanted. The residual lignin was washed with water (1×5.0 mL), the mixture was centrifuged again, and the supernatants were combined. The crude pHBA was obtained by evaporation of water at room temperature and the residue was dried under vacuum to obtain a brown solid of crude pHBA (8.5 mg, 8.5 wt %).

Production of pHBA from poplar wood powder: The NM6 poplar wood powder (5.0 g) was saponified using the same procedure as for the NM6 poplar lignin only at 90° C. for 3 h to obtain purified pHBA (78.5 mg, 1.6 wt %).

Production of pHBA from oil palm EFB powder: The oil palm EFB powder (1.76 g) was added into a 250 mL round-bottom flask containing a magnetic stir bar and fitted with a rubber stopper. Aqueous 0.2 M NaOH (105 mL) was added and the reaction mixture was stirred at 90° C. for 3 h. After the mixture was adjusted from pH ˜12 to PH ˜1 using concentrated HCl. The mixture was centrifuged (8,000 rpm) at room temperature for 10 min, and the supernatant was decanted. After washing the residual lignin with water (2×20 mL), the mixture was centrifuged again, and the supernatants were combined. The crude pHBA was extracted from the supernatants using EtOAc (3×100 mL), and the organic layers were combined, washed with brine (1×50 mL), dried over anhydrous sodium sulfate (Na₂SO₄), and filtered. Upon evaporation of the solvent in vacuo, a white solid was subjected to column chromatography on silica gel with eluent EtOAc to obtain purified pHBA (24.0 mg, 1.4 wt %).

Production of p-Hydroxybenzamide from Ethyl p-Hydroxybenzoate-Model Reaction:

Determination of the ratio of [pHBAm]:[pHBA] from the production of pHBAm from ethyl p-hydroxybenzoate is shown in Scheme 1. The ethyl p-hydroxybenzoate (100 mg) was added into a 25 mL glass pressure vessel containing a magnetic stir bar. Aqueous 29 wt % ammonium hydroxide (10 mL) was added to the vessel and the reaction mixture was stirred at the desired temperature in a sand bath for 24 h. After the mixture was cooled to room temperature, a 10 drop aliquot was transferred to a 10 mL flask and dried in vacuo. The crude product was analyzed by NMR spectroscopy: pHBAm 1H NMR (500 MHZ, DMSO-d₆) δ 6.75 (d, 2H, J=8.6 Hz, H_{3/5}), 7.71 (d, 2H, J=8.6 Hz, H_{2/6}), 9.94 (s, 1H, Ar—OH). pHBA 1H NMR (500 MHZ, DMSO-d₆) δ 6.80 (d, 2H, J=8.6 Hz, H_{3/5}), 7.77 (d, 2H, J=8.6 Hz, H_{2/6}), 10.20 (s, 1H, Ar—OH). Ratios of [pHBAm]:[pHBA] were determined by 1H NMR analysis of crude product in DMSO-d₆. The production of pHBAm was favored at temperatures >100° C.

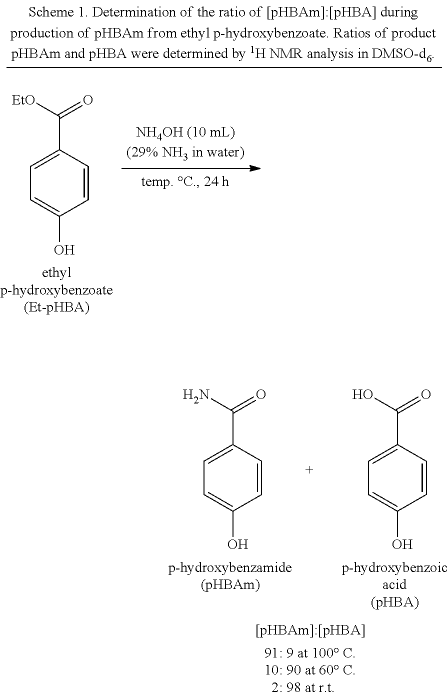 [SMILES: CCOC(=O)c1ccc(O)cc1 ; NC(=O)c1ccc(O)cc1 ; O=C(O)c1ccc(O)cc1]

Determination of the [pHBAm]:[pHBA] Ratio from NM6 Poplar Lignin:

The NM6 poplar lignin (100 mg) was added into the reaction vessel containing a magnetic stir bar. Aqueous 29 wt % ammonium hydroxide (10 mL) was added, and the reaction mixture was heated to the target temperature (25, 40, 60, 80, 100, 120, 130, or 140° C.) in a water (or oil) bath for 24 h. After the mixture was cooled to room temperature, a 10 drop aliquot was transferred to a 10 mL flask, were dried in vacuo. The crude product was analyzed by NMR spectroscopy. Ratios of [pHBAm]:[pHBA] were determined by ¹H NMR analysis of crude product in DMSO-d₆. The production of pHBAm was favored at temperatures >100° C. (Table 1).

Production of p-Hydroxybenzamide (pHBAm) from Biomass and Lignin:

Production of pHBAm from NM6 poplar lignin: The NM6 poplar lignin (200 mg) was added into a 25 mL stainless steel pressure vessel containing a magnetic stir bar. Aqueous 29 wt % ammonium hydroxide (10 mL) was added, and the reaction mixture was stirred at 140° C. in oil bath for 24 h. The mixture was then cooled to room temperature and adjusted from pH ˜12 to PH ˜1 using concentrated HCl at 0° C. The mixture was centrifuged (8,000 rpm) at room temperature for 10 min, and the supernatant was decanted off. After washing the residual lignin with RO water (1×20 mL), the mixture was centrifuged again, and RO water supernatants were combined. The crude pHBAm was extracted using EtOAc (3×20 mL), and the organic layers were combined, washed with brine (1×20 mL), dried over anhydrous sodium sulfate (Na₂SO₄), and filtered. Upon evaporation of solvent in vacuo, the resulting brown solid (31.0 mg) was subjected to preparative plate chromatography on silica gel with eluent EtOAc to obtain purified pHBAm (6.1 mg, 3.1 wt %). ¹H NMR (500 MHZ, DMSO-d₆) δ 6.75 (d, 2H, J=8.6 Hz, H_{3/5}), 7.71 (d, 2H, J=8.6 Hz, H_{2/6}), 9.94 (s, 1H, Ar—OH). ¹³C NMR (175 MHz, DMSO-d₆) δ 167.67 (C₇), 160.13 (C₄), 129.46 (C_{2/6}), 124.97 (C₁), 114.67 (C_{3/5}).

Production of pHBAm from poplar powder: The NM6 poplar powder (0.5 g) was treated using the same procedure as for the NM6 poplar lignin to obtain purified pHBAm (2.7 mg, 0.54 wt %).

Production of pHBAm from oil palm EFB powder: The oil palm EFB powder (0.5 g) was treated using the same procedure as for the NM6 poplar lignin to obtain purified pHBAm (5.9 mg, 1.2 wt %).

### Synthesis of Paracetamol (1) from p-Hydroxybenzamide in Batch Reactor—without Phenolic Group Protection

Synthesis of paracetamol (1) from pHBAm in two-step one-pot synthesis is shown in Scheme 2. The pHBAm (103.8 mg, 0.757 mmol, 1.0 eq.) was added into a 25 mL round-bottom flask fitted with a magnetic stir bar. Aqueous 1.0 M NaOH (4 mL) was added to dissolve the pHBAm at room temperature. Aqueous sodium hypochlorite (NaOCl, ˜13 wt %, 1.008 mmol, 1.3 eq.) was added dropwise at 0° C. and reaction mixture was stirred at room temperature for 1 h, then heated to 75° C. for 30 min, and then placed in an ice bath and cooled down to 0° C. Acetic acid (AcOH, 1 mL) was added dropwise to the mixture at 0° C., lowering the pH to ˜3. Then acetic anhydride (Ac₂O, 0.1 mL) was added dropwise to the mixture at 0° C. and the reaction was stirred at room temperature for 1 h. The mixture was filtered through Celite 535 (washed with EtOAc) and solvents were evaporated in vacuo to produce a brown oil. Purification by flash-column chromatography on silica gel (eluent EtOAc) gave 1 as a white solid (25.5 mg, 22.4%). ¹H NMR (500 MHZ, DMSO-d₆) δ 1.96 (s, 3H, C(O)CH₃), 6.65 (d, 2H, J=8.9 Hz, H_{3/5}), 7.32 (d, 2H, J=8.9 Hz, H_{2/6}), 9.13 (s, 1H, NH), 9.64 (s, 1H, Ar—OH). ¹³C NMR (125 MHz, DMSO-d₆) δ 167.50 (C═O), 153.10 (C₄), 131.04 (C₁), 120.76 (C_{2/6}), 114.97 (C_{3/5}), 23.76 (—CH₃); and recovered pHBAm (24.9 mg, 24.2%).

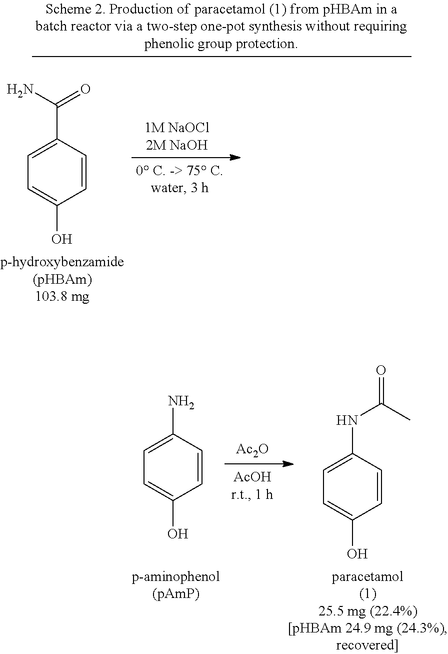

### Synthesis of Paracetamol (1) from p-Hydroxybenzoic Acid—with Phenolic Group Protection

The synthesis of paracetamol (1) from pHBA in which the phenolic group had been protected with MOM is shown in Scheme 3. The procedure protects the pHBAm and p-aminophenol from degradation during the synthesis of paracetamol.

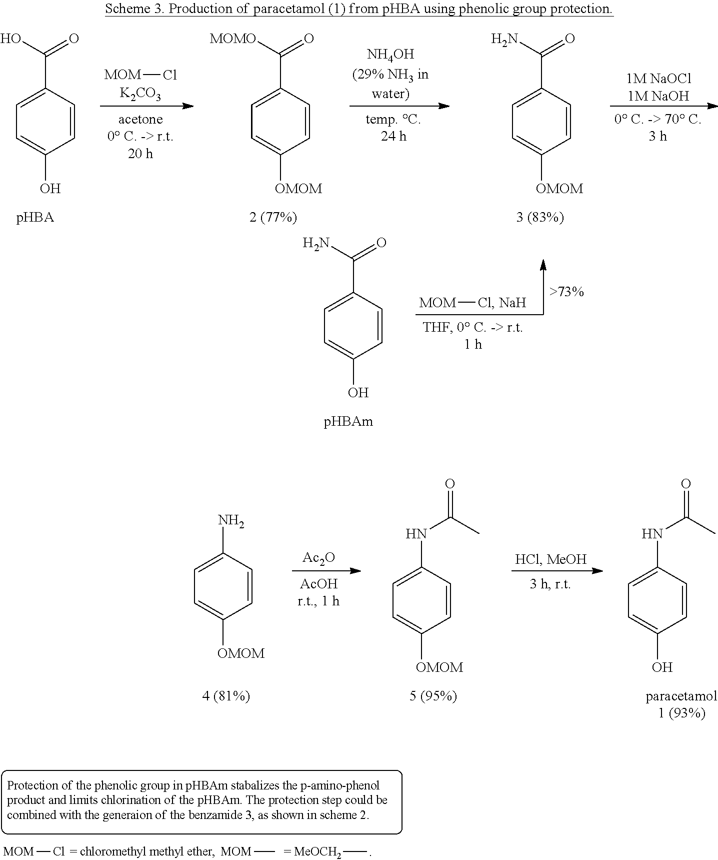

Synthesis of methoxymethyl 4-(methoxymethoxy)benzoate (2): The pHBA (992 mg, 7.182 mmol, 1.0 eq.), potassium carbonate (K₂CO₃, 4.483 g, 32.413 mmol, 4.5 eq.) and dry acetone (30 mL) were combined in a 250 mL flask fitted with a magnetic stir bar, and cooled to 0° C. in an ice water bath. Chloromethyl methyl ether (MOMCl, 1.2 mL, 15.801 mmol, 2.2 eq.) was added dropwise to the 0° C. reaction mixture. The reaction was warmed to room temperature and stirred for 20 h. The mixture was filtered, and acetone was evaporated in vacuo to produce of crude product 2. Purification by flash-column chromatography on silica gel (eluent EtOAc:hexanes=10:90 to 20:80) gave 2 as a colorless oil (1.25 g, 77%). ¹H NMR (700 MHZ, acetone-d₆) δ 8.01 (d, 2H, J=9.0 Hz, H_{2/6}), 7.13 (d, 2H, J=9.0 Hz, H_{3/5}), 5.43 (s, 2H, CH₂), 5.29 (s, 2H, CH₂), 3.49 (s, 3H, CH₃), 3.44 (s, 3H, CH₃). ¹³C NMR (175 MHz, acetone-d₆) δ 165.84 (C₇), 162.30 (C₄), 132.31 (C_{2/6}), 124.13 (C₁), 116.68 (C_{3/5}), 94.86 (—OCH₂O—), 91.22 (—OCH₂O—), 57.56 (—OCH₃), 56.31 (—OCH₃).

Synthesis of 4-(methoxymethoxy)benzamide (3). Compound 2 (99.5 mg, 0.4398 mmol) was added into a 60 mL glass pressure vessel containing a magnetic stir bar. Aqueous 29 wt % ammonium hydroxide (10 mL) was added, the vessel was sealed with the PTFE cap, and the reaction mixture was stirred at room temperature for 23 h. The mixture was transferred to a 125 mL separatory funnel and partitioned between EtOAc (20 mL) and water (20 mL). The organic layer was separated from the water and then the water layer was extracted with EtOAc (2×20 mL). The combined organics were washed with brine (1×20 mL), dried over Na₂SO₄, concentrated, and dried in vacuo to give 3 as a white solid (66.0 mg, 83%). ¹H NMR (700 MHZ, acetone-d₆) δ 7.89 (d, 2H, J=8.9 Hz, H_{2/6}), 7.06 (d, 2H, J=8.9 Hz, H_{3/5}), 5.25 (s, 2H, CH₂), 3.43 (s, 3H, CH₃). ¹³C NMR (125 MHZ, acetone-d₆) δ 168.26 (C₇), 160.63 (C₄), 130.07 (C_{2/6}), 128.63 (C₁), 116.28 (C_{3/5}), 94.80 (—OCH₂O—), 56.15 (—OCH₃).

Alternative method to synthesize 4-(methoxymethoxy)benzamide (3): Compound 3 was also prepared by dissolving pHBAm (4.72 g, 34.4 mmol, 1.0 eq.) and MOMCl (2.91 g, 36.1 mmol, 1.05 eq.) in THF (60 mL) and cooling the reaction to 0° C. in an ice bath. The reaction was purged with argon. Sodium hydride (60% NaH in mineral oil, 1.1 eq., 1.513 g, 37.8 mmol) was then added to the reaction and the mixture was gradually allowed to warm to room temperature. A white precipitate (sodium chloride, NaCl) was observed in the reaction flask. The reaction was quenched by filtration through qualitative filter paper, and the NaCl precipitate was washed with EtOAc (20 mL). The organic fractions were combined, and the solvents were removed to give a crude solid 3 (5.379 g). Recrystallization from EtOAc gave compound 3 as a white solid (4.53 g, 73% yield, first flush of crystals). 1H NMR (700 MHZ, acetone-d₆) δ 7.89 (d, 2H, J=8.9 Hz, H_{2/6}), 7.06 (d, 2H, J=8.9 Hz, H_{3/5}), 5.25 (s, 2H, CH₂), 3.43 (s, 3H, CH₃).

Synthesis of 4-(methoxymethoxy) aniline (4): Compound 3 (130.8 mg, 0.722 mmol, 1.0 eq.) was added into a 25 mL round-bottom flask fitted with a magnetic stir bar. Aqueous 1.0 M NaOH (4 mL, 3.970 mmol, 5.5 eq.) was added to dissolve the 3 at room temperature. Aqueous sodium hypochlorite (NaOCl, ˜13 wt %, 1.0 mL, 1.588 mmol, 2.2 eq.) was added dropwise at 0° C. and the reaction mixture was stirred for 55 min at 0° C., then heated to 70° C. at which temperature it was held for 3 h. The mixture was transferred in 250 ml separatory funnel and partitioned between EtOAc (30 mL) and H₂O (30 mL). The organic layer was separated from the water and then the water layer was extracted with EtOAc (2×30 mL). The combined organics were washed with brine (1×20 mL), dried over Na₂SO₄, filtered, concentrated by evaporation of solvents, and dried in vacuo to give crude 4 as a brown oil. Purification by flash-column chromatography on silica gel (eluent EtOAc:hexanes=50:50) gave 4 as a brown solid (89.9 mg, 81%). ¹H NMR (500 MHZ, CDCl₃) δ 6.85 (d, 2H, J=8.9 Hz, Ar), 6.61 (d, 2H, J=8.9 Hz, Ar), 5.06 (s, 2H, CH₂), 3.46 (s, 3H, CH₃). ¹³C NMR (125 MHz, CDCl₃) δ 150.42 (C₄), 141.42 (C₁), 118.06 (C_{2/6}), 116.44 (C_{3/5}), 95.70 (—OCH₂O—), 56.03 (—OCH₃).

Synthesis of N-[4-(methoxymethoxy)phenyl] acetamide (5): Compound 4 (42.9 mg, 0.280 mmol, 1.0 eq.) was added into a 50 mL round-bottom flask fitted with a magnetic stir bar. Acetic acid (AcOH, 2 mL) was added to dissolve the 4 at room temperature. Acetic anhydride (Ac₂O, 0.032 mL, 0.336 mmol, 1.2 eq.) was added at room temperature and the reaction mixture was stirred for 1 h. The mixture was concentrated by evaporation of solvents and dried in vacuo to give crude 5 as a brown solid. Purification by flash-column chromatography on silica gel (eluent EtOAc:hexanes=60:40 to 70:30) gave 5 as a white solid (51.9 mg, 95%). ¹H NMR (500 MHZ, CDCl₃) δ 7.37 (d, 2H, J=9.1 Hz, Ar), 6.98 (d, 2H, J=9.1 Hz, Ar), 5.12 (s, 2H, CH₂), 3.45 (s, 3H, CH₃O), 2.14 (s, 3H, C(O)CH₃. ¹³C NMR (125 MHz, CDCl₃) δ 168.31 (C═O), 154.22 (C₄), 132.18 (C₁), 121.93 (C_{2/6}), 116.95 (C_{3/5}), 94.91 (—OCH₂O—), 56.17 (—OCH₃), 24.67 (—CH₃).

Synthesis of paracetamol (1): Compound 5 (50.9 mg, 0.261 mmol) was added into a 50 mL round bottom flask with magnetic stir bar. The methanol (MeOH, 3 mL) was added to dissolve of 5 at room temperature. The concentrated hydrochloric acid (HCl, 0.3 mL) was added to the flask. The reaction mixture was stirred at room temperature and monitored by TLC for 3 h. After the compound 5 was consumed the mixture was concentrated by evaporation of solvents and dried in vacuo gave crude 1 as a white solid. Purification by flash-column chromatography on silica gel (eluent EtOAc) gave 1 as a white solid (36.8 mg, 93%) 1H NMR (500 MHZ, methanol-d₄) δ 7.30 (d, 2H, J=8.9 Hz, Ar), 6.73 (d, 2H, J=8.9 Hz, Ar), 2.08 (s, 3H, C(O)CH₃). ¹³C NMR (125 MHZ, methanol-d₄) δ 171.56 (C₇), 155.44 (C₄), 131.77 (C₁), 123.52 (C_{2/6}), 116.32 (C_{3/5}), 23.64 (—CH₃).

Continuous-Flow Reaction Design to Determine Optimal Ratio [NaOCl]:[pHBAm]:

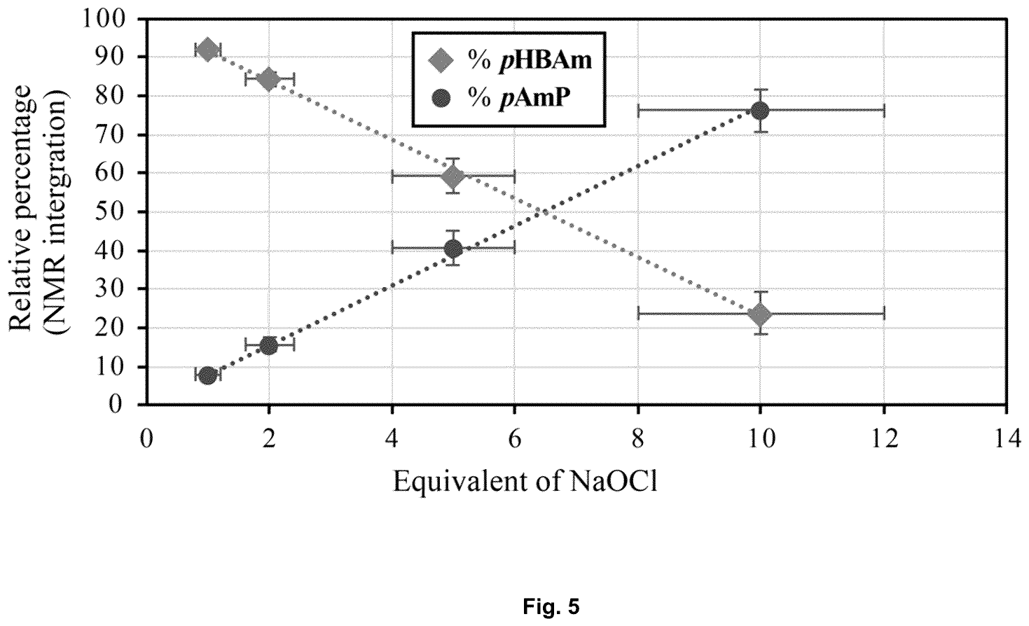

To achieve better control over the reaction conditions the process was transitioned into a continuous-flow reaction setup. This addressed issues with the batch reaction setup: 1) Suppressed the hypochlorite chlorination of the phenolic compounds; 2) Reduced the reaction time that the p-aminophenol product spent in the alkaline solution. Other advantages of the setup were that it allowed for easier tuning of reaction conditions (reaction time, reaction temperature at each stage of the setup, [NaOCl]:[pHBAm] ratio, quenching solutions, and improved scalability). FIG. 5 plots how the ratio of [NaOCl]- to -[pHBAm] impacts the conversion of pHBAm to p-aminophenol.

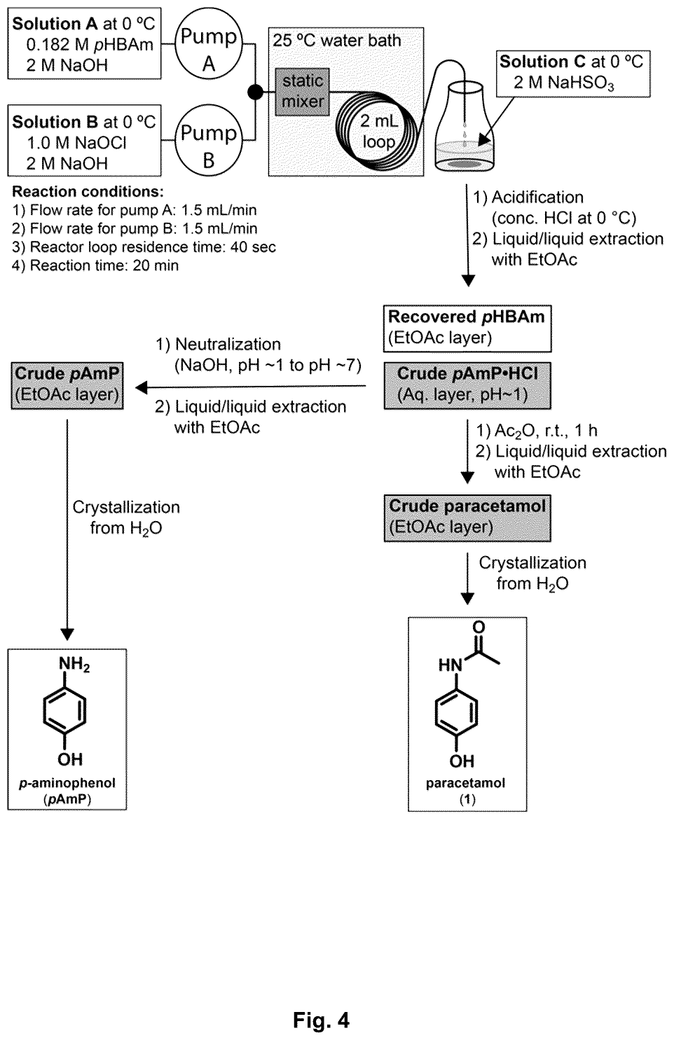

Several different configurations of the reactor design were tested. We found that the reaction proceeded similarly at 0° C., 25° C., and 50° C. The ratio of [NaOCl]:[pHBAm] and residence time of the p-aminophenol product in the pH ˜13 solution impacted the amount of chlorination, bleaching, condensation, and other side-reactions. The reaction is dependent on the pH of the reaction mixture and we found that 2 M NaOH gave a better yield of paracetamol than 1 M NaOH. The optimal adjustment of NaOH concentration was not preformed. Quenching of the reaction with sodium bisulfite (NaHSO₃) or sodium thiosulfate (NaS₂O₃) targeted removal of NaOCl that was still an effective chlorinating reagent under acidic conditions. The use of NaHSO₃ also had the advantage of neutralizing the NaOH and bringing the pH to ˜6.5. The NaHSO₃ solution could be added in-line at in stoichiometric equivalence to the total base (NaOH+NaOCl); see FIG. 4. The mixture could alternatively be pumped into a reservoir with excess NaHSO₃. Addition of acetic anhydride to either the crude p-aminophenol solutions or the NaHSO₃-neutralized solution resulted in acylation of the amine group in p-aminophenol to give paracetamol, forming sodium acetate and acetic acid.

Reaction conditions to study the [NaOCl]:[pHBAm] ratio were performed by adjusting the ratio of flow rates for solutions A and B (see FIG. 4), keeping the total flow rate constant at 3 mL/min. Increasing the number of equivalents of hypochlorite increased the conversion of pHBAm to p-aminophenol. See FIG. 5. It also increased the formation of chlorinated side-products, p-aminophenol degradation products (dark colored side products). When the NaOCl concentration was too high (˜10 eq.), the product mixture was bleached to a colorless solution and no product was observed.

Continuous-Flow Reaction Design for Converting pHBAm to Paracetamol:

Integrating the purification steps into the reaction scheme further enabled control over the formation of side-products. By first neutralizing the NaOCl with NaHSO₃ and then adjusting the solution to pH ˜1 with hydrochloric acid (HCl), the p-aminophenol product becomes stable as a hydrophylic ammonium salt (pAmP·HCl). See FIG. 4. Under this condition, the unreacted pHBAm can be extracted with liquid/liquid extraction using common organic solvents (e.g., EtOAc). This allows for the recycling of the unreacted pHBAm and prevents it from being acylated by Ac₂O. Treatment of the purified pAmP·HCl salt with Ac₂O both acylates the pAmP to make paracetamol and neutralizes the pH ˜1 solution to pH ˜6. A second liquid/liquid extraction with common organic solvents (e.g., EtOAc) extracts the paracetamol. The resulting pH ˜6 aqueous stream can then be sent to wastewater treatment as it contains mostly acetic acid, sodium sulfate, sodium chloride, and possibly some residual sulfite.

What is claimed is:

1. A method to produce paracetamol from biomass-derived p-hydroxybenzoic acid (pHBA), the method comprising:
(a) providing pHBA sourced from biomass;
(b) converting at least a portion of the pHBA into p-hydroxybenzamide (pHBAm);
(c) converting at least a portion of the pHBAm into p-aminophenol (pAmP); and
(d) converting at least a portion of the pAmP into paracetamol.

2. The method of claim 1, wherein in step (b) at least a portion of the pHBA is first esterified and then contacted with ammonium hydroxide to yield pHBAm.

3. The method of claim 2, wherein at least a portion of the pHBA is esterified by contacting it with an alcohol.

4. The method of claim 1, wherein in step (d), at least a portion of the pAmP is converted into paracetamol by contacting the pAmP with acetic anhydride.

5. The method of claim 1, wherein in step (c), at least a portion of the pHBAm is converted into pAmP using a Hofmann rearrangement reaction.

6. The method of claim 5, wherein in step (d), at least a portion of the pAmP is converted into paracetamol by contacting the pAmP with acetic anhydride.

7. The method of claim 5, wherein in step (c) the Hofmann rearrangement reaction yields pAmP and unreacted pHBAm, and further comprising separating at least a portion of the unreacted pHBAm from the pAmP, and recycling the unreacted pHBAm into step (c).

8. The method of claim 7, wherein at least a portion of the unreacted pHBAm is separated from the pAmP via liquid-liquid extraction.

9. The method of claim 8, wherein the liquid-liquid extraction is conducted using water and ethyl acetate.

10. The method of claim 7, wherein in step (d), at least a portion of the pAmP is converted into paracetamol by contacting the pAmP with acetic anhydride.

11. The method of claim 1, wherein steps (b), (c) and (d) are conducted in a solvent comprising water or ethyl acetate (EtOAc).

12. A method to produce paracetamol from biomass-derived p-hydroxybenzoic acid (pHBA), the method comprising:
(a) providing pHBA sourced from biomass;
(b) appending a protecting group to at least a portion of phenolic hydroxyl groups in pHBA to yield phenolic-protected pHBA;
(c) converting at least a portion of the phenolic-protected pHBA into phenolic-protected p-hydroxybenzamide (pHBAm);
(d) converting at least a portion of the phenolic-protected pHBAm into phenolic-protected p-aminophenol (pAmP);
(e) converting at least a portion of the phenolic-protected pAmP into phenolic-protected paracetamol; and
(f) deprotecting the phenolic-protected paracetamol to yield paracetamol.

13. The method of claim 12, wherein in step (b) a methoxymethyl ether (MOM) protecting group is appended to at least a portion of phenolic hydroxyl groups in pHBA.

14. The method of claim 12, wherein in step (e), at least a portion of the phenolic-protected pAmP is converted into phenolic-protected paracetamol by contacting the phenolic-protected pAmP with acetic anhydride.

15. The method of claim 12, wherein in step (d), at least a portion the phenolic-protected pHBAm is converted into phenolic-protected pAmP using a Hofmann rearrangement reaction.

16. The method of claim 15, wherein in step (d), at least a portion of the phenolic-protected pAmP is converted into phenolic-protected paracetamol by contacting the pAmP with acetic anhydride.

17. The method of claim 15, wherein in step (d) the Hofmann rearrangement reaction yields phenolic-protected pAmP and unreacted phenolic-protected pHBAm, and further comprising separating at least a portion of the unreacted phenolic-protected pHBAm from the phenolic-protected pAmP, and recycling the unreacted phenolic-protected pHBAm into step (d).

18. The method of claim 17, wherein at least a portion of the unreacted phenolic-protected pHBAm is separated from the pAmP via liquid-liquid extraction.

19. The method of claim 18, wherein the liquid-liquid extraction is conducted using water and ethyl acetate.

20. The method of claim 17, wherein in step (e), at least a portion of the phenolic-protected pAmP is converted into phenolic-protected paracetamol by contacting the phenolic-protected pAmP with acetic anhydride.

21. The method of claim 12, wherein steps (b), (c) (d), (e), and (f) are conducted in a solvent comprising water or ethyl acetate (EtOAc).
